(19) United States
(12) Patent Application Publication
(10) Pub. No.: US 2024/0060893 A1
(43) Pub. Date: Feb. 22, 2024

(54) SENSOR, DETECTION METHOD, AND DETECTION APPARATUS

(71) Applicant: Murata Manufacturing Co., Ltd., Nagaokakyo-shi (JP)

(72) Inventors: Yuka TOKUDA, Nagaokakyo-shi (JP); Shota USHIBA, Nagaokakyo-shi (JP); Madoka NISHIO, Nagaokakyo-shi (JP)

(21) Appl. No.: 18/386,356

(22) Filed: Nov. 2, 2023

Related U.S. Application Data
(63) Continuation of application No. PCT/JP2022/010212, filed on Mar. 9, 2022.

(30) Foreign Application Priority Data
May 11, 2021 (JP) 2021-080380

(51) Int. Cl. G01N 21/64

(52) U.S. Cl. CPC G01N 21/6428; G01N 2021/6439

(57) ABSTRACT

A biosensor includes a sensor element and probe molecules immobilized on a surface of the sensor element. The probe molecules are modified with a labeling substance to estimate an amount of the immobilized probe molecules on the sensor element.

### CROSS REFERENCE TO RELATED APPLICATIONS

This application claims the benefit of priority to Japanese Patent Application No. 2021-080380 filed on May 11, 2021 and is a Continuation application of PCT Application No. PCT/JP2022/010212 filed on Mar. 9, 2022. The entire contents of each application are hereby incorporated herein by reference.

### BACKGROUND OF THE INVENTION

### 1. Field of the Invention

The present invention relates to a sensor such as a biosensor, a pH sensor, or an ion sensor. The present invention also relates to a detection method and a detection apparatus which use the sensor.

### 2. Description of the Related Art

There is a demand for a highly sensitive detection method which can quickly detect a substance to be detected (also called target molecules) such as a cell, a microorganism, a virus, a protein, a nucleic acid (DNA or RNA), a peptide, an amino acid, or ions.

Sensors using probe molecules which specifically interact with target molecules have been proposed in recent years. Known examples of such sensors include a field effect transistor (FET)-type sensor, a quartz crystal microbalance (QCM)-type sensor, and a surface plasmon resonance (SPR)-type sensor.

In a sensor as described above, probe molecules are immobilized on the surface of a sensor element. A sensor element as used herein also includes a portion (e.g., a substrate, an electrode, etc.) other than a sensing portion. M. M. L. M. Vareiro, et al., Anal. Chem. 77, 2426 (2005) and X. Zhao, et al., Langmuir 27, 7654 (2011) state that the reactivity of probe molecules with target molecules is not in a simple proportional relationship with the amount of the immobilized probe molecules on a sensor element. When the amount of the immobilized probe molecules is too large, the reactivity with the target molecules is sometimes reduced by steric hindrance. Since the reactivity of probe molecules with target molecules affects the sensitivity of the sensor, it can be said that the amount of immobilized probe molecules is important for the sensing sensitivity.

K. Gajos, et al., Applied Surface Science 518, 146269 (2020) describes a method for analyzing the amount of immobilized probe molecules using ellipsometry, time-of-flight secondary ion mass spectrometry (TOF-SIMS), or atomic force microscopy (AFM).

### SUMMARY OF THE INVENTION

However, the analysis method described in K. Gajos, et al., Applied Surface Science 518, 146269 (2020) has the following problems.
Ellipsometry: Complex modeling is required.
TOF-SIMS: The method needs to be performed in high vacuum; therefore, a measurement sample can be destroyed. Further, a complicated analysis is required.
AFM: Because of a narrow measurement range, it undesirably takes time to perform an evaluation over a wide range of the order of several square millimeters.

As described above, the conventional method for evaluating the amount of immobilized probe molecules has the problems of being not suitable for wide-range evaluation, involving a complicated measurement or analysis, limitations placed on the makeup, shape, etc. of a measurement sample, etc.

Preferred embodiments of the present invention provide sensors, each of which can estimate an amount of immobilized probe molecules on a sensor element easily in a short time. Preferred embodiments of the present invention also provide detection methods and detection apparatuses each of which use the sensor.

A sensor according to a preferred embodiment of the present invention includes a sensor element and probe molecules immobilized on a surface of the sensor element, wherein the probe molecules are modified with a labeling substance to estimate an amount of immobilized probe molecules on the sensor element.

A detection method according to a preferred embodiment of the present invention is a method for detecting a to-be-detected substance using a sensor according to a preferred embodiment of the present invention, the detection method including supplying a sample including the to-be-detected substance to the sensor, measuring, in the sensor, a physical quantity which varies depending on an interaction between the to-be-detected substance and the probe molecules of the sensor, and correcting an output value of the sensor, obtained from the physical quantity, using a calibration curve indicating the relationship between the amount of the immobilized probe molecules and the reactivity of the probe molecules with the to-be-detected substance, which has been prepared in advance from known amounts of the probe molecules, and based on the amount of the immobilized probe molecules in the sensor, estimated from the labeling substance modifying the probe molecules of the sensor.

A detection apparatus according to a preferred embodiment of the present invention is an apparatus to detect a to-be-detected substance using a sensor according to a preferred embodiment of the present invention, the apparatus including a measurement section to measure, in the sensor, a physical quantity which varies depending on an interaction between the to-be-detected substance and the probe molecules of the sensor, and a correction section to correct an output value of the sensor, obtained from the physical quantity, using a calibration curve indicating the relationship between the amount of the immobilized probe molecules and the reactivity of the probe molecules with the to-be-detected substance, which has been prepared in advance from known amounts of the probe molecules, and based on the amount of the immobilized probe molecules in the sensor, estimated from the labeling substance modifying the probe molecules of the sensor.

According to preferred embodiments of the present invention, it is possible to provide sensors each of which can estimate the amount of immobilized probe molecules on a sensor element easily in a short time. According to preferred embodiments of the present invention, it is also possible to provide detection methods and detection apparatuses using a sensor according to a preferred embodiment of the present invention.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a schematic diagram showing an example of a biosensor according to a first preferred embodiment of the present invention.

FIG. 2 is a schematic diagram showing an example of a covalent bond using an amino group of a probe molecule.

FIG. 3 is a schematic diagram showing an example of a covalent bond using a sulfhydryl group of a probe molecule.

FIG. 4 is a schematic diagram showing an example of a biosensor according to a third preferred embodiment of the present invention.

FIG. 5 is a schematic diagram showing an example of a biosensor according to a fourth preferred embodiment of the present invention.

FIG. 6 is a schematic diagram showing another example of the biosensor according to the fourth preferred embodiment of the present invention.

FIG. 7 is a schematic diagram showing an example of a biosensor according to a fifth preferred embodiment of the present invention.

FIG. 8 is a diagram showing an example of the structure of a linker molecule.

FIG. 9 is a schematic diagram showing an example of a biosensor according to a sixth preferred embodiment of the present invention.

FIG. 10 is a schematic diagram showing an example of a biosensor according to a seventh preferred embodiment of the present invention.

FIG. 11 is a schematic diagram illustrating an example of how a biosensor is used.

FIG. 12 is a graph showing relationships between gate voltage V_{G} and source-drain current I_{DS}.

FIG. 13 is a diagram illustrating a step of modifying a probe molecule with a labeling substance.

FIG. 14 is a diagram illustrating a step of producing a graphene FET device.

FIG. 15 is a diagram illustrating a step of forming an oxide film.

FIG. 16 is a diagram illustrating a step of performing a silane coupling treatment.

FIG. 17 is a diagram illustrating a step of immobilizing labeled probe molecules.

FIG. 18 shows fluorescence micrographs at varying inputs of labeled probe molecules.

### DETAILED DESCRIPTION OF THE PREFERRED EMBODIMENTS

Sensors, detection methods, and detection apparatuses according to preferred embodiments of the present invention will now be described.

It is to be noted that the present invention is not limited to the preferred embodiments described below, and changes may be made as appropriate to the preferred embodiments without departing from the spirit and scope of the present invention. Two or more preferable features as described below with reference to the preferred embodiments may be combined, and such a combined feature is within the scope of the present invention.

Preferred embodiments of sensors of the present invention will be described with reference to biosensors as examples. It should be noted that sensors according to preferred embodiments of the present invention can also be applied to sensors other than biosensors, for example, pH sensors or ion sensors.

The below-described preferred embodiments are by way of example only. Partial substitutions or combinations of features according to different preferred embodiments are of course possible. For the second and subsequent preferred embodiments, only differences from the first preferred embodiment will be described while omitting duplicate descriptions. In particular, the same effect achieved by the same construction will not be described for every relevant preferred embodiment.

### First Preferred Embodiment

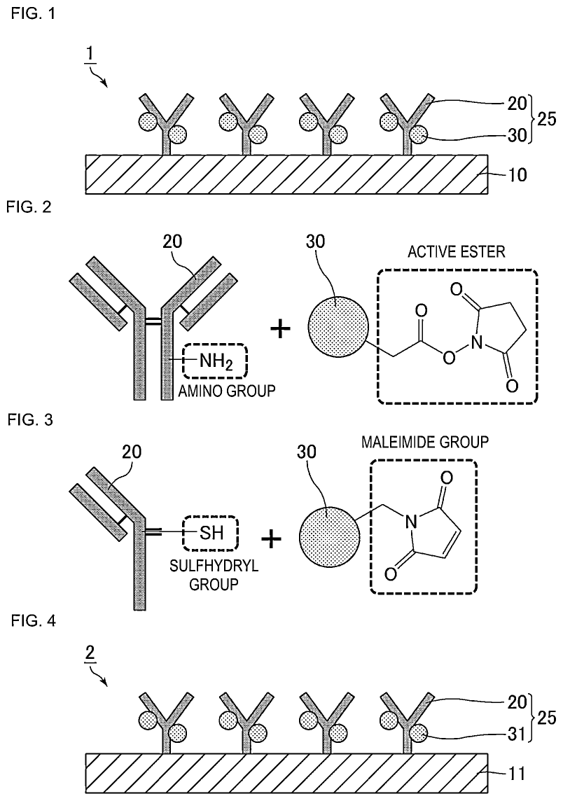

FIG. 1 is a schematic diagram showing an example of a biosensor according to the first preferred embodiment of the present invention.

In FIG. 1, the construction of the biosensor is modified as appropriate for clarity and simplification of the drawing. The same holds true for other drawings.

The biosensor 1 shown in FIG. 1 includes a sensor element 10, and probe molecules 20 immobilized on the surface of the sensor element 10. The probe molecules 20 are modified with a labeling substance 30. Thus, labeled probe molecules 25, which are the probe molecules 20 modified with the labeling substance 30, are immobilized on the surface of the sensor element 10.

In a sensor according to a preferred embodiment of the present invention, examples of the probe molecules immobilized on the surface of the sensor element include an antibody, an enzyme, a sugar chain, an aptamer (nucleic acid), a lectin, an oligonucleotide, a peptide, and a low-molecular-weight organic polymer. Among them, at least one selected from the group consisting of an antibody, an enzyme, a peptide, and a lectin is preferred. The probe molecules may be mobile with some degree of freedom as long as they remain on the surface of the sensor element.

Compared to the conventional evaluation methods, sensors according to preferred embodiments of the present invention can estimate the amount of the immobilized probe molecules on the sensor element easily in a short time by detecting the labeling substance modifying the probe molecules. In particular, sensors according to preferred embodiments of the present invention are expected to achieve the advantages that a wide-range evaluation can be performed in a short time, that an evaluation can be performed without setting complicated measurement conditions or performing a complicated analysis, and that an evaluation can be performed while keeping a sample in a sensible state without processing the sample.

Further, the probe molecules after modification with the labeling substance have an increased molecular weight. Accordingly, the van der Waals force increases, facilitating immobilization of the probe molecules on the surface of the sensor element.

In addition, it is possible to control the hydrophilicity/hydrophobicity of the surface of the probe molecules as well as the amount of charge and the sign of the charge on the surface by the type of the labeling substance. This enables the probe molecules to easily capture target molecules, thus increasing the sensitivity.

For example, when hydrophilicity is imparted to the probe molecules, non-specific adsorption is suppressed by the hydrophilic surface, resulting in increased sensitivity. On the other hand, when hydrophobicity is imparted to the probe molecules, their hydrophobic interaction with target molecules increases. This enables the probe molecules to easily capture the target molecules, thus increasing the sensitivity.

Further, by controlling the amount of charge on the probe molecules, it is possible to increase the electrostatic attractive force acting between the probe molecules and target molecules or to decrease the electrostatic repulsive force acting therebetween. This enables the probe molecules to easily capture the target molecules, thus increasing the sensitivity.

### Second Preferred Embodiment

In the second preferred embodiment of the present invention, the probe molecules are modified with the labeling substance via a covalent bond. Because of the formation of the stable covalent bond, the labeling substance is unlikely to be detached from the probe molecules.

For example, an amino group (—NH₂), a sulfhydryl group (—SH), or a carboxy group (—COOH) of a probe molecule can be used to form a covalent bond with the labeling substance. In this case, a functional group present at the end of an amino acid of a probe molecule may be used. For example, an amino group present at the end of a lysine, a sulfhydryl group present at the end of a cysteine, or a carboxy group present at the end of aspartic acid or glutamic acid can be used.

FIG. 2 is a schematic diagram showing an example of a covalent bond using an amino group of a probe molecule.

For example, as shown in FIG. 2, a covalent bond is formed between an amino group of each probe molecule 20 and an active ester of the labeling substance 30.

FIG. 3 is a schematic diagram showing an example of a covalent bond using a sulfhydryl group of a probe molecule.

For example, as shown in FIG. 3, a covalent bond is formed between a sulfhydryl group of each probe molecule 20 and a maleimide group of the labeling substance 30.

When a carboxy group of a probe molecule is used, a covalent bond is formed between it and an amino group of the labeling substance by, for example, activating the carboxy group with an activating agent such as EDC/NHS.

### Third Preferred Embodiment

In the third preferred embodiment of the present invention, the sensor element is a field effect transistor (FET) device. Further, the labeling substance is a fluorescent dye, a fluorescent protein, quantum dots, or gold nanoparticles.

FIG. 4 is a schematic diagram showing an example of a biosensor according to the third preferred embodiment of the present invention.

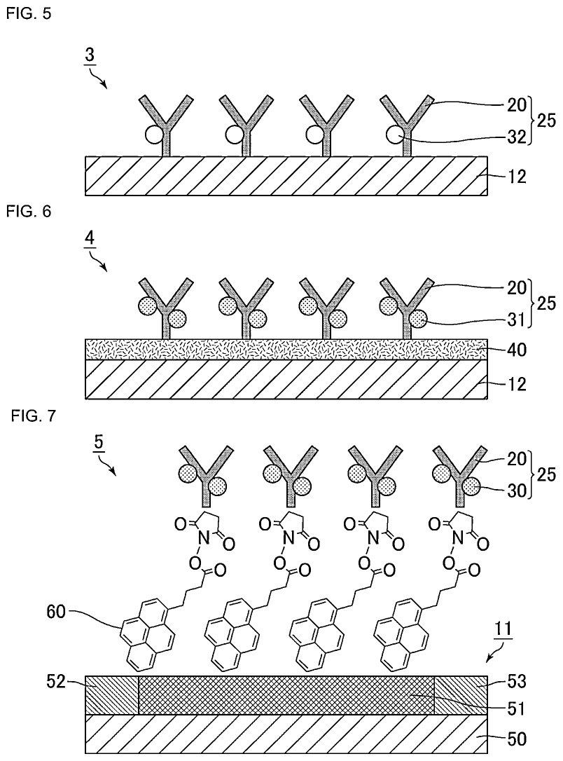

The biosensor 2 shown in FIG. 4 includes an FET device 11 and probe molecules 20 immobilized on the surface of the FET device 11. The probe molecules 20 are modified with a fluorescent dye or fluorescent protein 31. Thus, labeled probe molecules 25, which are the probe molecules 20 modified with the fluorescent dye or fluorescent protein 31, are immobilized on the surface of the FET device 11. The probe molecules 20 may be modified with quantum dots or gold nanoparticles 32 (see FIG. 5) instead of the fluorescent dye or fluorescent protein 31.

A fluorescent dye, a fluorescent protein, and quantum dots can be observed using a fluorescence microscope, while gold nanoparticles can be observed using a dark field microscope. Therefore, by modifying probe molecules with a fluorescent dye, a fluorescent protein, quantum dots, or gold nanoparticles as a labeling substance, a time-dependent change in the amount of the immobilized probe molecules can be estimated easily in a short time using a fluorescence microscope, a dark field microscope, or an optical microscope.

An FET-type biosensor is configured to form a mechanism which simulates a living body in a channel portion, and detects a reaction that occurs there using an electrical property of the FET. When the sensor element is an FET device, immobilization of probe molecules modified with a labeling substance on the surface of the FET device makes it possible to adjust the amount of doping in a semiconductor and the surface potential, thereby increasing the sensitivity.

For example, the semiconductor is p-doped when an amino group of a probe molecule is modified, while the semiconductor is n-doped when a carboxy group of a probe molecule is modified.

The closer the surface potential is to 0 mV, the higher the sensitivity. The surface potential can be measured with a zeta potential measuring device.

The fluorescent dye is a fluorescent label having a molecular weight on the order of not more than 10,000. Examples of the fluorescent dye include FITC (fluorescein isothiocyanate), Alexa Fluor (registered trademark) dye, and Cy (registered trademark) dye. The fluorescent protein is a fluorescent label having a molecular weight on the order of not less than 10,000. Examples of the fluorescent protein include PE (Phycoerythrin) and APC (Allophycocyanin).

The size of the quantum dots is, for example, not less than several nanometers and not more than several tens of nanometers. The particle size of the gold nanoparticles is, for example, not less than several tens of nanometers and not more than several hundred nanometers.

### Fourth Preferred Embodiment

In the fourth preferred embodiment of the present invention, the sensor element is a quartz crystal microbalance (QCM) device or a surface plasmon resonance (SPR) device.

FIG. 5 is a schematic diagram showing an example of a biosensor according to the fourth preferred embodiment of the present invention.

The biosensor 3 shown in FIG. 5 includes a QCM device or SPR device 12 and probe molecules 20 immobilized on the surface of the QCM device or SPR device 12. The probe molecules 20 are modified with quantum dots or gold nanoparticles 32. Thus, labeled probe molecules 25, which are the probe molecules 20 modified with the quantum dots or gold nanoparticles 32, are immobilized on the surface of the QCM device or SPR device 12.

When the sensor element is a QCM device or an SPR device, by modifying probe molecules with quantum dots or gold nanoparticles as a labeling substance, a time-dependent change in the amount of the immobilized probe molecules can be estimated easily in a short time using a fluorescence microscope, a dark field microscope, or an optical microscope.

FIG. 6 is a schematic diagram showing another example of the biosensor according to the fourth preferred embodiment of the present invention.

The biosensor 4 shown in FIG. 6 includes a QCM device or SPR device 12 and probe molecules 20 immobilized on the surface of the QCM device or SPR device 12. The probe molecules 20 are immobilized on the surface of the QCM device or SPR device 12 via an insulating film 40. The probe molecules 20 are modified with a fluorescent dye or fluorescent protein 31.

When the sensor element is a QCM device or an SPR device, by modifying probe molecules with a fluorescent dye or a fluorescent protein as a labeling substance, a time-dependent change in the amount of the immobilized probe molecules can be estimated easily in a short time using a fluorescence microscope.

Further, the provision of an insulating film on the surface of the sensor element makes it possible to suppress fluorescence quenching.

The insulating film is, for example, an oxide film which will be described below with reference to the sixth preferred embodiment.

### Fifth Preferred Embodiment

In the fifth preferred embodiment of the present invention, the sensor element includes an FET device, and the FET device includes graphene or carbon nanotubes. Probe molecules are immobilized on the surface of the FET device via linker molecules each having a pyrenyl group. Besides such linker molecules each having a pyrenyl group, it is possible to use linker molecules which each have a benzene ring and which are capable of n-n interaction.

FIG. 7 is a schematic diagram showing an example of a biosensor according to the fifth preferred embodiment of the present invention.

The biosensor 5 shown in FIG. 7 includes an FET device 11 and probe molecules 20 immobilized on the surface of the FET device 11. The probe molecules 20 are modified with a labeling substance 30. Thus, labeled probe molecules 25, which are the probe molecules 20 modified with the labeling substance 30, are immobilized on the surface of the FET device 11.

The FET device 11 includes an insulating substrate 50, a semiconductor layer 51 disposed on the insulating substrate 50, and a source electrode 52 and a drain electrode 53, both disposed on the insulating substrate 50 and electrically connected to the semiconductor layer 51. The source electrode 52 and the drain electrode 53, disposed on the insulating substrate 50, are located apart from each other. The insulating substrate 50 is exposed between the source electrode 52 and the drain electrode 53. In the example illustrated in FIG. 7, the semiconductor layer 51 is disposed on the insulating substrate 50 such that it covers the exposed area of the insulating substrate 50. The semiconductor layer 51 may be disposed on the insulating substrate 50 such that it covers an end portion of the source electrode 52, the exposed area of the insulating substrate 50, and an end portion of the drain electrode 53. The semiconductor layer 51 between the source electrode 52 and the drain electrode 53 defines the channel of the FET device 11.

The insulating substrate 50 is, for example, a thermally oxidized silicon substrate obtained by oxidizing the surface of a silicon (Si) substrate to form a silicon oxide (SiO₂) layer, or a boron nitride (BN) substrate. There is no particular limitation on the material of the insulating substrate 50. Examples of usable materials include inorganic compounds such as silicon oxide, silicon nitride, aluminum oxide, titanium oxide, and calcium fluoride; and organic compounds such as an acrylic resin, a polyimide, and a fluorocarbon resin. There is no particular limitation on the shape of the insulating substrate 50; it may be a flat-plate shape or a curved-plate shape. The insulating substrate 50 may have flexibility.

The semiconductor layer 51 includes graphene or carbon nanotubes. The use of the FET-type transistor having the graphene or carbon nanotube channel can provide a sensor having an increased sensitivity.

Graphene is a two-dimensional material including carbon atoms bonded in a hexagonal network. Graphene has a very large specific surface area (surface area per volume) and has a very high electrical mobility.

Graphene generally refers to a carbonaceous sheet-like material including a monolayer of carbon atoms in a honeycomb structure. As used herein, however, graphene has a broader meaning including the following materials:
A carbonaceous sheet-like material in which graphene is multi-layered or partially multi-layered up to 100 layers
A carbonaceous sheet-like material which is polycrystalline and has grain boundaries, which is partially broken, and which has an end portion(s)
A carbonaceous sheet-like material which is partially elementally substituted, or in which the honeycomb structure is partially collapsed
Graphene oxide and reduced graphene oxide obtained by reducing it
Ribbon-shaped (strip-shaped) graphene
Carbon nanotube composed of sheet-like graphene in a tubular shape, and a material composed of graphene in a rolled shape

The carbon nanotube is a long tubular carbon compound. A single-wall carbon nanotube (SW-CNT) including a single carbon layer having the same network structure as that of graphene, for example, may be used as the carbon nanotube.

The number of layers of the semiconductor layer 51 is not limited to one and may be two or three or more. The number of layers of the semiconductor layer 51 is preferably 10 or less, more preferably 5 or less. The number of layers need not be uniform throughout the semiconductor layer 51. For example, a single-layer portion and a multi-layer portion may be mixed. The number of layers of the semiconductor layer 51 can be measured by, for example, Raman spectroscopy or cross-sectional observation using a transmission electron microscope (TEM).

The source electrode 52 and the drain electrode 53 are, for example, electrodes each having a multilayer structure including a titanium (Ti) layer and a gold (Au) layer. Besides titanium and gold, a metal such as gold, platinum, titanium, or palladium may be used as an electrode material in a single layer, or two or more such metals may be used in a multilayer structure.

Linker molecules 60 are disposed on the surface of the semiconductor layer 51. The probe molecules 20 are immobilized on the surface of the FET device 11 via the linker molecules 60 each having a pyrenyl group.

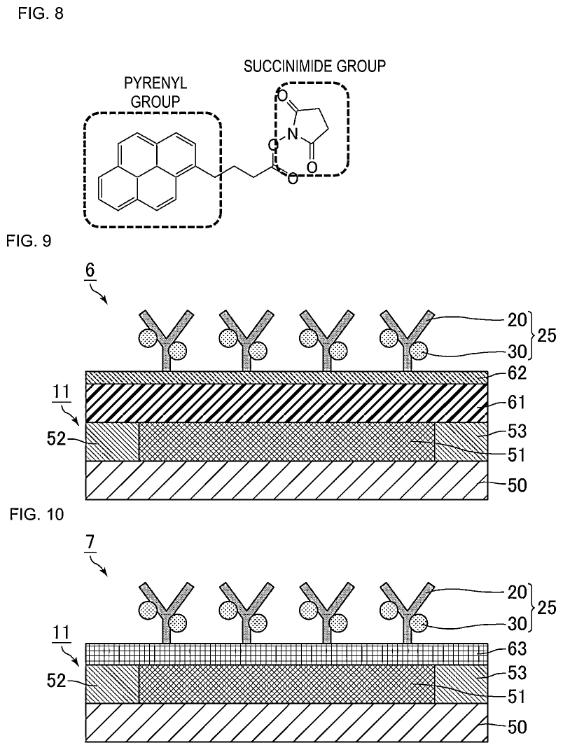

FIG. 8 is a diagram showing an example of the structure of a linker molecule.

FIG. 8 shows 1-pyrenebutanoic acid succinimidyl ester (PBASE) as an example of a linker molecule having a pyrenyl group.

As shown in FIG. 8, a succinimide group is present at one end of a PBASE. Therefore, a probe molecule can be immobilized by binding the linker molecule to an amino group of the probe molecule. A pyrenyl group, including four benzene rings arranged two-dimensionally, exists at the other end of the PBASE. Therefore, the PBASE can be bound to the surface of a carbonaceous semiconductor layer such as graphene by a non-covalent bond called n-n interaction. Accordingly, compared to the case where the probe molecules are fixed on the surface of the semiconductor layer by a covalent bond, the effect on the crystal structure of the semiconductor layer can be reduced. This can reduce a deterioration of the properties.

Further, by detecting the labeling substance modifying the probe molecules, it is possible to check whether or not the carbonaceous semiconductor layer is broken. In particular, when the labeling substance is a fluorescent dye, a fluorescent protein, quantum dots, or gold nanoparticles, it is possible to visually check whether or not the carbonaceous semiconductor layer is broken.

### Sixth Preferred Embodiment

In the sixth preferred embodiment of the present invention, the sensor element is an FET device, and the FET device includes graphene or carbon nanotubes. Probe molecules are immobilized on the surface of the FET device via an oxide film. By covering the surface of the semiconductor layer with the oxide film, the surface of the semiconductor layer is made hydrophilic. This suppresses non-specific adsorption, resulting in increased sensitivity.

In the sixth preferred embodiment of the present invention, probe molecules are preferably immobilized on the surface of the FET device via a silane coupling agent present on the surface of the oxide film. When the silane coupling agent is present on the surface of the oxide film, the oxide film and the silane coupling agent, and the silane coupling agent and the probe molecules are firmly bound together respectively by a covalent bond. Therefore, the probe molecules are stably immobilized, resulting in increased sensitivity.

FIG. 9 is a schematic diagram showing an example of a biosensor according to the sixth preferred embodiment of the present invention.

In the biosensor 6 shown in FIG. 9, an oxide film 61 is disposed on the surface of the semiconductor layer 51, and a silane coupling agent 62 is disposed on the surface of the oxide film 61. Probe molecules 20 modified with a labeling substance 30 (i.e., labeled probe molecules 25) are immobilized on the surface of the FET device 11 via the oxide film 61 and the silane coupling agent 62. The silane coupling agent 62 on the surface of the oxide film 61 may be omitted. In that case, the probe molecules 20 modified with the labeling substance 30 (i.e., the labeled probe molecules 25) may be immobilized on the surface of the FET device 11 via the oxide film 61.

In the example illustrated in FIG. 9, the oxide film 61 is disposed entirely on the insulating substrate 50 such that it covers the surface of the semiconductor layer 51. While the oxide film 61 may be disposed also on the source electrode 52 and the drain electrode 53 as shown in FIG. 9, it suffices if the oxide film 61 is disposed on the insulating substrate 50 such that it covers at least the surface of the semiconductor layer 51.

Examples of the oxide of the oxide film 61 include SiO₂, Al₂O₃, TiO₂, HfO₂, ZrO₂, SiNₓ, and a composite oxide thereof.

The oxide of the oxide film 61 can be confirmed by subjecting the sensor surface to elemental analysis by X-ray photoelectron spectroscopy (XPS). Alternatively, the oxide can be confirmed by subjecting the sensor surface to elemental analysis by energy dispersive X-ray spectroscopy (EDS).

Examples of methods for forming the oxide film 61 include vapor deposition, sputtering, atomic layer deposition (ALD), thermal chemical vapor deposition (thermal CVD), and catalytic chemical vapor deposition (catalytic CVD).

The thickness of the oxide film 61 is preferably about 2 nm or more, for example, from the viewpoint of securing the electrical insulation of the sensor surface and ensuring the mechanical stability of the oxide film 61 (e.g., mechanical stability against ultrasonic cleaning). On the other hand, the thickness of the oxide film 61 is preferably about 30 nm or less, for example. When the thickness of the oxide film 61 is about 30 nm or less, for example, high sensitivity of the sensor can be secured.

The thickness of the oxide film 61 can be measured by cross-sectional observation with a transmission electron microscope (TEM).

The oxide film 61 preferably includes an amorphous region. Compared to the case where the entire oxide film 61 is crystalline, the electrical insulation of the sensor surface can be enhanced. When the oxide film 61 includes an amorphous region, the oxide film 61 need not be amorphous in its entirety and may partially include a crystalline region.

The inclusion of an amorphous region in the oxide film 61 can be confirmed by crystallinity analysis of an X-ray diffraction image or an electron beam diffraction image in transmission electron microscope (TEM) measurement.

When the silane coupling agent 62 is present on the surface of the oxide film 61, the silane coupling agent 62 may be, for example, a silane coupling agent having an amino group, such as 3-aminopropyltriethoxysilane (APTES) or 3-aminopropyltrimethoxysilane (APTMS), a silane coupling agent having a thiol group, such as 3-mercaptopropyltriethoxysilane (MPTES), or a silane coupling agent having an epoxy group, such as triethoxy(3-glycidyloxypropyl)silane (GPTES).

The presence of the silane coupling agent 62 on the surface of the oxide film 61 can be confirmed by surface analysis using time-of-flight secondary ion mass spectrometry (TOF-SIMS).

The silane coupling agent 62 can be replaced with another material as long as the material forms a covalent bond on the oxide film 61. A phosphonic acid derivative is an example of such a material.

In the biosensor according to the sixth preferred embodiment of the present invention, the probe molecules may be immobilized on the surface of the FET device via spacer molecules present on the surface of the oxide film.

When the spacer molecules are present on the surface of the oxide film, the probe molecules are separated from the surface of the oxide film by the spacer molecules and thus obtain a degree of freedom, leading to an enhancement of the sensing ability of the probe molecules. Further, when the spacer molecules have hydrophilicity, the hydrophilicity of the sensor surface can be enhanced.

Examples of the spacer molecules include polyethylene glycol (PEG), polyvinylpyrrolidone (PVP), dextran, and ethylene glycol bis(succinimidyl succinate). The arm length of the spacer molecules is desirably not less than about 0.7 nm and not more than about 10 nm, for example, but is not limited thereto.

In the biosensor according to the sixth preferred embodiment of the present invention, a seed layer may be provided between the semiconductor layer and the oxide film. The provision of the seed layer can form the oxide film uniformly, resulting in increased sensitivity.

The seed layer can be formed, for example, by forming a film of a single-component metal, e.g., a light metal such as aluminum (Al) or magnesium (Mg), a 3d transition metal such as titanium (Ti), nickel (Ni), or chromium (Cr), or a rare metal such as hafnium (Hf), zirconium (Zr), or yttrium (Y), and then oxidizing the metal film.

The thickness of the seed layer is preferably about 2 nm or less, for example. On the other hand, the thickness of the seed layer is preferably about 0.5 nm or more, for example.

In the biosensor according to the sixth preferred embodiment of the present invention, the oxide film may have surface irregularities. The provision of the irregularities in the surface of the oxide film can increase the surface area of the oxide film, thereby enhancing the hydrophilicity of the sensor surface. Further, the provision of the surface irregularities can increase the density of the probe molecules, resulting in increased sensitivity.

Examples of methods for forming the irregularities in the surface of the oxide film include a method which involves roughening the surface of the oxide film by surface blasting or plasma asking, a method which involves forming the oxide film by island growth, etc. Island growth is a phenomenon in which nuclei, spaced apart from each other, grow from the nuclei as starting points. A film is formed non-uniformly by island growth, and irregularities are formed in the surface of the film. For example, the wettability of a surface of an underlying film, on which a film is to be grown, for the material of the film is required to be poor in order to cause island growth of the film.

### Seventh Preferred Embodiment

In the seventh preferred embodiment of the present invention, a blocking agent is present, together with the probe molecules, on the FET device. The blocking agent enhances the hydrophilicity of the sensor surface to thereby suppress non-specific adsorption, resulting in increased sensitivity.

FIG. 10 is a schematic diagram showing an example of a biosensor according to the seventh preferred embodiment of the present invention.

In the biosensor 7 shown in FIG. 10, labeled probe molecules 25, which are probe molecules 20 modified with a labeling substance 30, are immobilized on the surface of an FET device 11. A blocking agent 63 is present, together with the probe molecules 20, on the FET device 11.

Examples of the blocking agent 63 include a protein (e.g., bovine serum albumin (BSA), hemoglobin, or skimmed milk), a surfactant (e.g., Tween (trade name), Triton (trade name), or sodium dodecyl sulfate (SDS)), and a polymer (e.g., PEG or PVP). These blocking agents may be used singly or in a combination of two or more.

In a sensor according to a preferred embodiment of the present invention, when the sensor element is an FET device, the FET device may further include a gate electrode to externally apply an electric field to the semiconductor layer.

An FET device operates, for example, in a liquid. In that case, the surface of the FET device, which is to contact the liquid, has a site capable of binding to a substance to be detected.

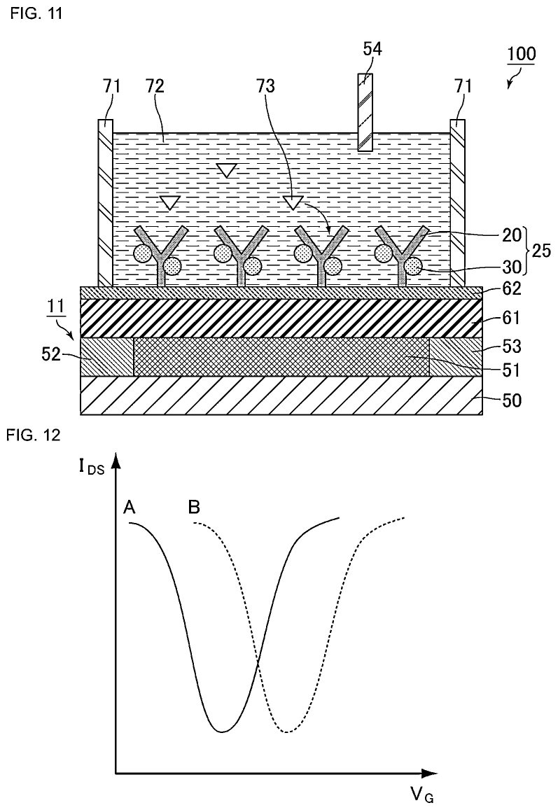

FIG. 11 is a schematic diagram illustrating an example of how a biosensor is used.

As with the biosensor 6 shown in FIG. 9, the biosensor 100 shown in FIG. 11 includes an FET device 11 and probe molecules 20 immobilized on the surface of the FET device 11. The probe molecules 20 are modified with a labeling substance 30. The FET device 11 includes an insulating substrate 50, a semiconductor layer 51 disposed on the insulating substrate 50, and a source electrode 52 and a drain electrode 53, both disposed on the insulating substrate 50 and electrically connected to the semiconductor layer 51. An oxide film 61 is disposed on the surface of the semiconductor layer 51, and a silane coupling agent 62 is disposed on the surface of the oxide film 61. The probe molecules 20 modified with the labeling substance 30 (i.e., labeled probe molecules 25) are immobilized on the surface of the FET device 11 via the oxide film 61 and the silane coupling agent 62.

In the biosensor 100 shown in FIG. 11, a pool fence 71 made of, for example, silicone rubber is mounted on the FET device 11. The space inside the pool fence 71 is filled with an electrolytic solution 72, and a gate electrode 54 is immersed in the electrolytic solution 72. A bipotentiostat (not shown) is connected to the source electrode 52, the drain electrode 53, and the gate electrode 54. The electrolytic solution 72 includes a to-be-detected substance (target molecules) 73.

The gate electrode 54 is to apply a potential to the source electrode 52 and the drain electrode 53 and is generally made of a noble metal. The gate electrode 54 is provided at a position other than the positions where the source electrode 52 and the drain electrode 53 are formed. The gate electrode 54 is generally provided on the insulating substrate 50 or at a location other than the location of the insulating substrate 50 and is preferably provided above the source electrode 52 or the drain electrode 53.

FIG. 12 is a graph showing relationships between gate voltage V_{G} and source-drain current I_{DS}.

In FIG. 12, the solid line A indicates the source-drain current I_{DS} as observed when probe molecules are not bound to a to-be-detected substance, and the dashed line B indicates the source-drain current I_{DS} as observed when the probe molecules are bound to the to-be-detected substance. As shown in FIG. 12, when the probe molecules specifically bind to the to-be-detected substance, the conduction characteristics are modulated by the charge of target molecules, the to-be-detected substance. By observing the modulation, it is possible to sense the presence/absence or concentration of the to-be-detected substance.

A sensor element for use in a sensor according to a preferred embodiment of the present invention is not limited to a sensor element which electrically detects the specific binding of probe molecules to a to-be-detected substance. It is possible to use a sensor element which detects the specific binding optically or mechanically.

Sensors of preferred embodiments of the present invention are not limited to the above-described preferred embodiments, and various applications and variations of the construction of the sensors, the production conditions, etc. may be made within the scope of the present invention.

For example, a sensor according to a preferred embodiment of the present invention may include an insulating coating layer provided in an area other than the sensing area of a sensor element. The provision of the insulating coating layer enhances the insulating properties of that area other than the sensing area, thus enhancing the reliability of the sensor. Further, target molecules are no longer captured in the area other than the sensing area, leading to increased sensitivity of the sensor.

The material of the insulating coating layer is, for example, an organic compound such as polyimide, an epoxy resin, an acrylic resin, or a fluorocarbon resin. The thickness of the insulating coating layer is desirably not less than about 100 nm and not more than about 10 μm, for example.

Alternatively, when the sensor element is an FET device, an insulating coating layer may be provided on a source electrode and a drain electrode, and a semiconductor layer may be disposed on the source electrode, the drain electrode, and the insulating coating layer.

A method for detecting a to-be-detected substance using a sensor according to a preferred embodiment of the present invention will now be described.

In advance of measurement, the amount of immobilized probe molecules in the sensor is estimated by detecting a labeling substance modifying the probe molecules of the sensor. Further, from known amounts of the probe molecules, a calibration curve indicating the relationship between the amount of the immobilized probe molecules and the reactivity of the probe molecules with a to-be-detected substance is prepared in advance.

A sample including the to-be-detected substance is supplied to the sensor to bring the sample into contact with the sensor element. When the sample including the to-be-detected substance is a liquid, the sample may be dropped onto the sensor element using a dropper or the like, or the sample may be introduced to the sensor element using a flow channel.

Examples of the sample including the to-be-detected substance include a body fluid such as saliva, throat swab, nasal mucus, tear fluid, or blood of a subject, a biological sample such as urine or feces, a suspension of cells or viruses themselves, drinking water, sewage, and breath. The sample including the to-be-detected substance need not necessarily be a liquid.

Subsequently, a physical quantity, which varies depending on an interaction between the to-be-detected substance and the probe molecules of the sensor, is measured in the sensor.

The physical quantity to be measured differs depending on the type of the sensor element. The physical quantity is, for example, an electrical physical quantity or an optical physical quantity. Examples of the electrical physical quantity include voltage value, current value, frequency, electrical resistance value, and conductivity. Examples of the optical physical quantity include light wavelength, light intensity, light reflectance, and light transmittance.

For example, when the sensor element is an FET device, the sensor measures the value (drain current value) of an electric current that flows between a source electrode and a drain electrode when a voltage is applied between the source electrode and the drain electrode.

An output value of the sensor, obtained from the physical quantity, is corrected using the calibration curve indicating the relationship between the amount of the immobilized probe molecules and the reactivity of the probe molecules with the to-be-detected substance, which has been prepared in advance from known amounts of the probe molecules, and based on the amount of the immobilized probe molecules in the sensor, estimated from the labeling substance modifying the probe molecules of the sensor.

As described above, there is a correlation between the amount of probe molecules immobilized on a sensor element and the reactivity of the probe molecules with a to-be-detected substance. In view of this, from known amounts of the probe molecules, a calibration curve indicating the relationship between the amount of immobilized probe molecules and the reactivity of the probe molecules with the to-be-detected substance is prepared in advance. Using the calibration curve, an output value of the sensor obtained from the physical quantity, i.e., the amount of signal response, can be corrected based on the amount of the immobilized probe molecules in the sensor, estimated from the labeling substance modifying the probe molecules of the sensor. This can enhance the accuracy and reliability of measurement.

An apparatus for detecting a to-be-detected substance a sensor according to a preferred embodiment of the present invention is another preferred embodiment of the present invention.

A detection apparatus according to a preferred embodiment of the present invention is an apparatus to detect a to-be-detected substance using a sensor according to a preferred embodiment of the present invention, includes a measurement section to measure, in the sensor, a physical quantity which varies depending on an interaction between the to-be-detected substance and the probe molecules of the sensor, and a correction section to correct an output value of the sensor, obtained from the physical quantity, using a calibration curve indicating the relationship between the amount of the immobilized probe molecules and the reactivity of the probe molecules with the to-be-detected substance, which has been prepared in advance from known amounts of the probe molecules, and based on the amount of the immobilized probe molecules in the sensor, estimated from the labeling substance modifying the probe molecules of the sensor.

As with a detection method according to a preferred embodiment of the present invention, in advance of measurement, the amount of immobilized probe molecules in the sensor is estimated by detecting a labeling substance modifying the probe molecules of the sensor. The estimation of the amount of the immobilized probe molecules is preferably performed using an apparatus different from the detection apparatus of the present invention. Further, from known amounts of the probe molecules, a calibration curve indicating the relationship between the amount of the immobilized probe molecules and the reactivity of the probe molecules with a to-be-detected substance is prepared in advance.

### EXAMPLES

The following examples illustrate a biosensor as a sensor according to a preferred embodiment of the present invention in greater detail. It should be noted that the present invention is not limited to the examples.

First, a biosensor having the same construction as the biosensor 6 shown in FIG. 9 was produced by the following procedure.

### (1) Labeling of Probe Molecules

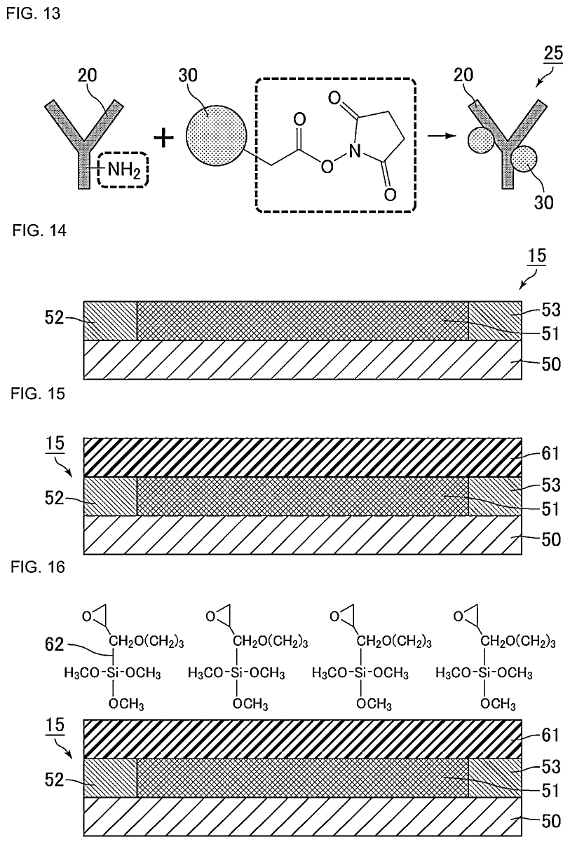

FIG. 13 is a diagram illustrating a step of modifying a probe molecule with a labeling substance. As shown in FIG. 13, a probe molecule 20 is modified with a labeling substance 30 by chemical bonding to produce a labeled probe molecule 25.

Specific example conditions are as follows.

Probe molecules: Anti-human CRP monoclonal antibody (Oriental Yeast Co., Ltd.) or Anti-Influenza A Virus Nucleoprotein monoclonal antibody (Bio Matrix Research, Inc.)

Labeling substance: Alexa Fluor 488 NHS Ester (Thermo Fisher Scientific)

### Labeling Procedure

1. The labeling substance (fluorescent dye) is dissolved in N,N-dimethylformamide (DMF) to prepare a solution having a concentration of 10 mg/mL.
2. The solution prepared in 1. above is dropped onto a 1 to 10 mg/mL solution of the above probe molecules (antibody), and the mixture is reacted at room temperature for one hour under stirring.
3. An unreacted labeling substance is removed by a desalting column.
4. A preservative (sodium azide) is added to the solution after modification with the labeling substance, and the solution is stored at 4° C.

### (2) Production of Graphene FET Device

FIG. 14 is a diagram illustrating a step of producing a graphene FET device. As shown in FIG. 14, a semiconductor layer 51, a source electrode 52, and a drain electrode 53 are formed on an insulating substrate 50 to produce a graphene FET device 15.

For example, a Ti layer and an Au layer are formed on the insulating substrate 50 using a method such as vacuum deposition, electron beam (EB) deposition, or sputtering. Thereafter, the source electrode 52 and the drain electrode 53 are formed by patterning using photolithography and etching. Subsequently, graphene that has been grown, for example, on a copper foil is transferred to the insulating substrate 50, followed by patterning using photolithography and etching to form the semiconductor layer 51 on the insulating substrate 50.

### (3) Formation of Oxide Film

FIG. 15 is a diagram illustrating a step of forming an oxide film. As shown in FIG. 15, an oxide film 61 is formed on the graphene FET device 15.

For example, the oxide film 61 made of silica, alumina, or a composite oxide thereof is formed using a method such as atomic layer deposition (ALD) or EB deposition.

### (4) Silane Coupling Treatment

FIG. 16 is a diagram illustrating a step of performing a silane coupling treatment. As shown in FIG. 16, the surface of the oxide film 61 is subjected to silane coupling treatment.

A gas phase method or a liquid phase method is used for the silane coupling treatment. In the example illustrated in FIG. 16, a GPTES is used as a silane coupling agent 62, and therefore epoxy groups are present on the surface.

### (5) Immobilization of Labeled Probe Molecules

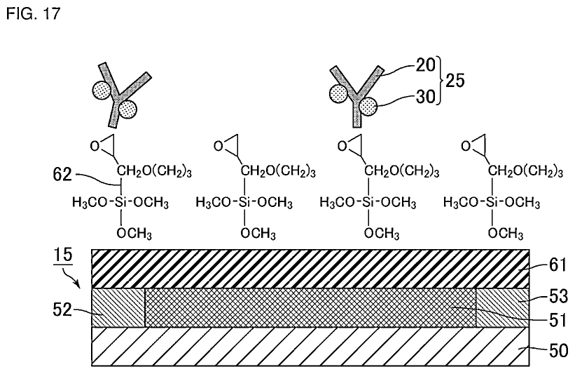

FIG. 17 is a diagram illustrating a step of immobilizing labeled probe molecules. As shown in FIG. 17, the labeled probe molecules 25 are immobilized on the surface of the graphene FET device 15.

For example, the solution of the labeled probe molecules 25, prepared in (1) above, is dropped onto the graphene FET device 15 which has undergone the silane coupling treatment described in (4) above.

The biosensor thus produced was observed by a fluorescence microscope. The observation conditions are, for example, 500-μm field of view, 20-fold objective lens, and exposure time 1 to 5 seconds.

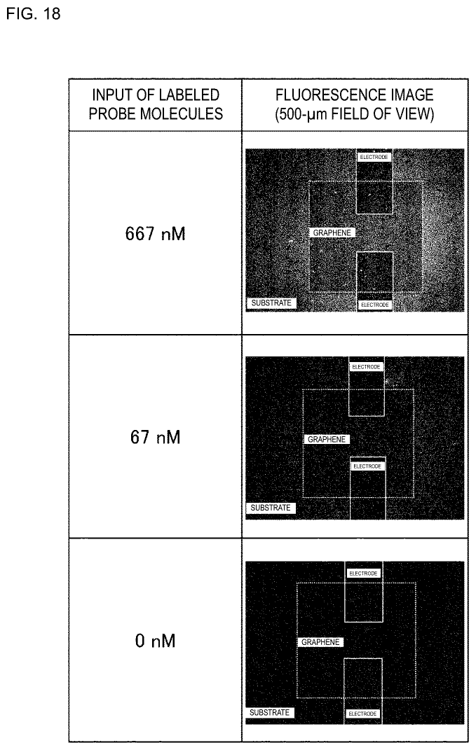

FIG. 18 shows fluorescence micrographs at varying inputs of the labeled probe molecules.

As can be seen in FIG. 18, fluorescence was not observed when the input of the labeled probe molecules is 0 nM, whereas fluorescence was observed when the input of the labeled probe molecules was 67 nM or 667 nM. FIG. 18 also indicates that the higher the input of labeled probe molecules, the stronger the fluorescence.

The results thus indicate that a biosensor having probe molecules modified with a fluorescent dye as a labeling substance can estimate the amount of the immobilized probe molecules by detecting the labeling substance using a fluorescence microscope.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A sensor comprising:
a sensor element; and
probe molecules immobilized on a surface of the sensor element; wherein
the probe molecules are modified with a labeling substance to estimate an amount of the immobilized probe molecules on the sensor element;
the sensor element includes a field effect transistor (FET) device.

2. The sensor according to claim 1, wherein the probe molecules are modified with the labeling substance via a covalent bond.

3. The sensor according to claim 1, wherein the labeling substance includes a fluorescent dye, a fluorescent protein, quantum dots, or gold nanoparticles.

4. The sensor according to claim 1, wherein the FET device comprises graphene or carbon nanotubes.

5. The sensor according to claim 4, wherein the probe molecules are immobilized on a surface of the FET device via linker molecules each including a pyrenyl group.

6. The sensor according to claim 4, wherein the probe molecules are immobilized on a surface of the FET device via an oxide film.

7. The sensor according to claim 6, wherein the probe molecules are immobilized on the surface of the FET device via a silane coupling agent on a surface of the oxide film.

8. The sensor according to claim 4, wherein a blocking agent is present, together with the probe molecules, on the FET device.

9. The sensor according to claim 1, wherein the probe molecules are at least one selected from the group consisting of an antibody, an enzyme, a peptide, and a lectin.

10. A detection method for detecting a to-be-detected substance using a sensor including a sensor element and probe molecules immobilized on a surface of the sensor element, the probe molecules being modified with a labeling substance to estimate an amount of immobilized probe molecules on the sensor element, the detection method comprising:
supplying a sample including the to-be-detected substance to the sensor;
measuring, in the sensor, a physical quantity which varies depending on an interaction between the to-be-detected substance and the probe molecules of the sensor; and
correcting an output value of the sensor, obtained from the physical quantity, using a calibration curve indicating a relationship between the amount of the immobilized probe molecules and reactivity of the probe molecules with the to-be-detected substance, which has been prepared in advance from known amounts of the probe molecules, and based on the amount of the immobilized probe molecules in the sensor, estimated from the labeling substance modifying the probe molecules of the sensor.

11. A detection apparatus for detecting a to-be-detected substance using a sensor including a sensor element and probe molecules immobilized on a surface of the sensor element, the probe molecules being modified with a labeling substance to estimate an amount of immobilized probe molecules on the sensor element, the detection apparatus comprising:
a measurement section to measure, in the sensor, a physical quantity which varies depending on an interaction between the to-be-detected substance and the probe molecules of the sensor; and
a correction section to correct an output value of the sensor, obtained from the physical quantity, using a calibration curve indicating a relationship between the amount of the immobilized probe molecules and reactivity of the probe molecules with the to-be-detected substance, which has been prepared in advance from known amounts of the probe molecules, and based on the amount of the immobilized probe molecules in the sensor, estimated from the labeling substance modifying the probe molecules of the sensor.

12. The detection apparatus according to claim 11, wherein the probe molecules are modified with the labeling substance via a covalent bond.

13. The detection apparatus according to claim 11, wherein the labeling substance includes a fluorescent dye, a fluorescent protein, quantum dots, or gold nanoparticles.

14. The detection apparatus according to claim 11, wherein the FET device comprises graphene or carbon nanotubes.

15. The detection apparatus according to claim 14, wherein the probe molecules are immobilized on a surface of the FET device via linker molecules each including a pyrenyl group.

16. The detection apparatus according to claim 14, wherein the probe molecules are immobilized on a surface of the FET device via an oxide film.

17. The detection apparatus according to claim 16, wherein the probe molecules are immobilized on the surface of the FET device via a silane coupling agent on a surface of the oxide film.

18. The detection apparatus according to claim 14, wherein a blocking agent is present, together with the probe molecules, on the FET device.

19. The detection apparatus according to claim 11, wherein the probe molecules are at least one selected from the group consisting of an antibody, an enzyme, a peptide, and a lectin.
